(19) United States
(12) Patent Application Publication
(10) Pub. No.: US 2015/0344839 A1
(43) Pub. Date: Dec. 3, 2015

(54) Components for Animal Feed and Use Thereof

(71) Applicant: University of Plymouth, Devon (GB)

(72) Inventors: Peter Brooks, Plymouth (GB); Jane Beal, Plymouth (GB); Soumelia Savvidou, Plymouth (GB); Vlasta Demeckova, Humernne (SK)

(73) Assignee: University of Plymouth, Devon (GB)

(21) Appl. No.: 14/823,284

(22) Filed: Aug. 11, 2015

Related U.S. Application Data
(63) Continuation of application No. 13/262,489, filed on Jan. 25, 2012.

(30) Foreign Application Priority Data
Mar. 31, 2009 (GB) 0905505.4
Mar. 31, 2009 (GB) 1119438.8

(51) Int. Cl. C12N 1/20; A23K 1/00; A23K 1/18; C12R 1/225

(52) U.S. Cl. CPC C12N 1/20; C12R 1/225; A23K 1/009; A23K 1/1826

(57) ABSTRACT

A fermented liquid feed composition for a target animal is provided, the feed composition being prepared by the fermentation of a feed substrate with a lactic acid producing bacteria, the lactic acid bacteria being characterised by being viable under the conditions prevailing in the gastrointestinal tract of the target animal, being an aggregating bacteria and/or co-aggregating with one or more pathogens, and being able to produce upon fermentation in the feed substrate lactic acid in an amount of at least a minimum inhibitory concentration of lactic acid. A method of producing a fermented liquid feed composition is also provided. Preferred lactic acid producing bacteria for use in the composition and method are Lactobacillus.

The present invention relates to feed materials for animals, to the provision of fermented animal feed products, methods for their preparation and the use thereof.

It is common practice to prepare animal feeds as dry compositions, that is up to 15% moisture, to avoid spoilage. The dry feed material may be stored for extended periods of time and transported with little or no degradation. However, the cost of preparing dried feed is increasing. In particular, in excess of 60% of the energy costs on preparing the dry feed are consumed in the actual drying stage. Accordingly, there is a growing need for an alternative to the known dry animal feeds.

One such alternative are moist or liquid feeds. In this regard, moist feeds, such as those fed to chickens and other poultry, contain up to 30% by weight of water. Liquid feeds, such as those fed to pigs, contain up to 70% by weight of water. Known for some time, liquid feeds can be difficult to formulate, prepare and store in a cost effective manner. In addition, moist or liquid feeds are difficult to store and transport over long distances. In particular, wet or liquid animal feeds are very prone to spoiling due to the growth of mould, bacteria and yeast, making the long term storage of wet and liquid animal feeds a difficult prospect. There is therefore a need to address the problem of storing and transporting wet and liquid feeds. It would also be most advantageous if the range of starting materials for preparing wet and liquid animal feeds could be extended. Currently, co-products and residues from industries such as dairy, bakery and distilling are used to prepare wet and liquid feed for animals. Raw materials with significant future potential are the co-products of bioethanal and biofuel production. These are already incorporated into diets following drying, which is an extremely energy demanding mode of treatment. It would be beneficial, if a way can be found to formulate these co-products into a moist or liquid feed. Other potential sources for raw materials for incorporation into moist and liquid animal feeds include human food grade residues from the slaughter process and from meat and fish processing. The value of much of this material is currently lost due to poor storage, drying or disposal to landfill. All these processes have a high energy demand and adverse environmental impact.

One improvement to the preparation of wet and liquid animal feeds is the inoculation of the feed raw material with one or more suitable microorganisms, to produce a so-called ‘fermented feed’. This process is synonymous with the process of ‘ensiling’, which is the ubiquitous method used for the preservation of herbage for feeding to ruminant animals. The inoculant is selected to inhibit the growth of mould, yeasts and spoilage bacteria that will propagate in and spoil the feed material. The process of producing fermented feed can be seen as a form of ‘biopreservation’. EP 0 906 952 discloses a bacterial strain for the ensiling of straw fodder. The strain, of the genus Lactococcus, was found to be effective in inhibiting the growth or yeast, clostridia, mould, gram positive bacteria and certain gram negative bacteria in the ensiling of green fodder.

Further, US 2002/0054935 is concerned with a livestock feed composition suitable for the fattening of livestock, such as cattle, goats, sheep, swine and fowl. The nutritional value of the livestock feed is increased by inoculation with one or more strains of Aspergillus. The livestock feed treated in this way consists of a fibrous feed material, a cereal, and an organic waste material. It appears that the Aspergillus is used to modify the nutrient content of the feed. However, this can have disadvantageous results, as many Aspergillus spp. produce mycotoxins harmful to many animals.

U.S. Pat. No. 6,403,084 is concerned with mixed cultures for improved fermentation and aerobic stability of silage. The problem of aerobic instability of silage is addressed, in particular the rapid growth of yeast and mould that can occur, resulting in the silage being spoiled. Further, it is noted that silage may be spoiled by the growth of yeast, even when inoculated and subjected to a good fermentation phase, in which microorganisms are used to ferment the silage and produce lactic acid, reducing the pH and giving rise to acid conditions. Acid-tolerant yeasts are considered to be responsible for the spoilage of fermented silage. As a solution to these problems. U.S. Pat. No. 6,403,084 proposes inoculating the silage with a combination of the homofermentative lactic acid bacteria Lactobacillus plantarum and the heterofermentative lactic acid bacteria Lactobacillus buchneri or Lactobacillus brevis. The aforementioned combination of microorganisms is alleged to provide sufficiently low pH conditions to preserve the silage and prevent spoiling due to the growth of mould and yeast.

WO 99/18188 describes a feed product for horses. The feed product comprises one or more strains of Lactobacillus having the ability to colonize the equine intestines. The microorganisms were isolated from the gastric or intestinal mucosa of horses.

GB 2,167,639 discloses a process for the treatment of industrial or agricultural waste matter, such as animal protein. The process involves chopping the waste as an aqueous mass and treating the resulting material with proteolytic enzymes to form a suspension, obtaining a gelatinised starch content in the suspension and adding to the suspension amylolytic enzymes and a lactic acid producing culture. The resulting mixture is fermented to produce simple sugars and lactic acid.

U.S. Pat. No. 4,214,985 relates to sewerage treatment. The treatment involves inoculating sewerage sludge with L. plantarum bacteria and a carbohydrate, such as lactose. The resulting mixture is fermented until the pH falls below 4.0. The thus produced composition is used as a soil extender.

JP 2007082468 is concerned with providing a microorganisms preparation for feed. The preparation comprises particular strains of Lactobacillus plantarum and/or Bacillus subtilis. Fermented feeds may be produced by adding the microorganisms to organic wastes, such as silage grass, and fermenting.

WO 89/05849 describes the selection of lactic acid bacteria isolated from the gastrointestinal tract of pigs for their ability to survive in the environment of the gastrointestinal tract and to adhere to the epithelium of the gastrointestinal tract of the target animal. The bacteria selected with these properties may be included in a fermented milk product for human consumption or in a veterinary composition for providing to pigs for the prevention or treatment of gastrointestinal diseases.

More recently, WO 2008/006382 discloses homofermented liquid animal feed products. As discussed in WO 2008/006382, the production of fermented animal feeds using microorganism-containing inoculants is very difficult, often leading to the fermented feed containing pathogenic bacteria, such as Vibrio spp., Campylobacter spp., Salmonella spp., E. coli, and Staphylococcus aureus. The fermented feed may also contain a high content of various yeasts and moulds. It is noted that the ingestion by the livestock of such inappropriately fermented feeds may result in morbidity and mortality. WO 2008/006382 notes that the sterile handling of bacteria required by farmers wishing to prepare their own fermented feed is often impossible to achieve. Further, there is a practice of using a continuous fermentation process, in which a portion of one batch of fermented feed is used as an inoculum for a subsequent fermentation batch. This leads to a gradual build up of harmful and undesirable microorganisms in the fermented feed. In an attempt to address these problems, WO 2008/006382 proposes a method for preparing a fermented mixed feed, the method comprising: providing a liquid fermented product; providing a feed product to be fermented; combining the aforementioned products; and fermenting the feed product using the liquid fermented product as an inoculum. A fermented feed prepared by this method is also described.

While proposals have been made to provide fermented feeds that are resistant to spoilage due to the growth of yeasts, moulds, bacteria and other organisms, there is still a need for an improved fermented feed that may be stored for extended periods of time and transported, without significant spoilage.

As mentioned in WO 2008/006382, a further issue relating to feedstuffs, in particular moist or liquid feed materials, is the health and wellbeing of the livestock consuming the feeds. As noted in WO 2008/006382, a poorly fermented feed may be a source of microorganisms harmful to the animals consuming the feed. More generally, animals are susceptible to a wide range of infections arising from microorganisms that enter and colonise the gastrointestinal (GI) tract of the animal. EP 0 955 061 addresses the issue of gastroenteric infections in pigs, in particular porcine rotavirus, porcine coronavirus, enterotoxigenic and enteropathogenic strains of Escherichia coli, Clostridium sp., Salmonella sp., Serpulina hyodysenteriae, Serpulina pilosicoli, Lawsonia intracellularis, Isospora suis, and Cryptosporidium. As a solution to the problem of gastroenteric infections in pigs. EP 0 955 061 proposes an oral product characterised by containing at least one specific antibody to the aforementioned microorganisms, derived from the egg yolks of immunized hens. It is noted in EP 0 955 061 that lactacidogenic bacteria administered to pigs can have a probiotic effect, suppressing the propagation of the enteropathogenic or enterotoxigenic bacteria and enhance the activity of the animal's immune system. Accordingly, a preferred embodiment of EP 0 955 061 includes one or more lactic acid bacteria, such as Enterococcus spp. and Lactobacillus spp.

As discussed in EP 0 955 061, young animals are particularly susceptible to infections of the GI tract, leading to illness and death. Ways of improving the health and wellbeing of finishing pigs are described by P. Brooks et al., ‘The Effect on Biological Performance and Faecal Microbiology of Feeding Finishing Pigs on Liquid Diets Fermented with Lactic Acid Bacteria’, SafePork, 2005, page 149. Brooks et al. note that fermented liquid feeds (FLF) have been shown to reduce the incidence of salmonella in pigs. In particular, it was found that a lactic acid concentration of 70 mMol/kg in the fermented feed exhibited bacteriostatic activity with respect to Salmonella spp., but higher concentrations of lactic acid in excess of 100 mMol/kg were needed to be bactericidal. However. Brooks et al. had found that natural fermentations had produced unpredictable results in commercial feed units and referred to a study that found that only 3% of commercial fermentations of wheat and barley produced more than 75 mMol/kg of lactic acid after 24 hours of fermentation. It was concluded that fermentations to produce lactic acid in high concentrations relying on indigenous microorganisms present in the grains could not be relied upon for commercial production of fermented feeds. Brooks et al. conducted experiments using specific LAB to examine the effect on biological performance and faecal microbiology of pigs fed diets of fermented liquid feeds. The results showed that the pigs retained good health when fed on the fermented liquid feed, showing no change in average daily weight gain when fed with the FLF compared with a standard feed. In addition, the experiments showed that, while the fermented feed contained lactic acid bacteria in high concentrations, the concentration of LAB in the faeces of the pigs remained unchanged. However, analysis of the faeces for the presence of coliforms indicated that the coliform content was reduced in the pigs fed with the FLF diet. This in turn indicated an improvement in the health of the pig and a lower risk of infection and illness. It was concluded that the selection of the LAB used for fermentation was important in achieving the reduction in coliforms.

As noted by Brooks et al., achieving a specific concentration of lactic acid in the fermented feed is important in achieving the beneficial effects of the fermented feeds. Techniques for measuring the concentrations of lactic acid in fermented feeds are described by S. J. Niven, et al., ‘The Simultaneous Determination of Short Chain Fatty Acid, Monosaccharides and Ethanol in Fermented Liquid Pig Diets’, Animal Feed Science and Technology, 117 (2004), pages 339 to 345.

The thesis of V. Demeckova, ‘Benefits of Fermented Liquid Diets for Sows and their Piglets’, Department of Agriculture and Food, Faculty of Land, Food and Leisure. University of Plymouth, July 2003, describes experiments conducted with liquid feed fermented with Lactobacillus plantarum to determine their effects on the antimicrobial and potential immunological effects on sows in late gestation periods. The results indicated that certain strains of Lactobacillus were both an effective inoculant for the preparation of fermented liquid feeds, as well as providing probiotic activity to the sow once the fermented feed was ingested. Significantly, immunoglobulin levels in the sows' colostrums were increased. Colostrum from sows fed fermented feed also increased the mitogenic activity of blood lymphocytes and enterocytes. These factors could in turn improve the resistance of the sows and their piglets to pathogen challenges.

Drago, L., et al., ‘Inhibition of in vitro growth of enteropathogens by new Lactobacillus isolates of human intestinal origin’, FEMS Microbiology Letters, 153 (1997), pages 455 to 463, describe experiments to isolate strains of Lactobacillus from the faeces of new born infant humans and examine their effect in co-cultures on certain pathogenic bacteria. The experiments conducted were entirely in vitro and, while showing some beneficial effects of the Lactobacillus strains in reducing the growth of certain pathogens, did not relate at all to the formulation of feeds for animals.

It would be most advantageous if a fermented liquid feed composition could be provided that may be prepared on a commercial scale from a wide range of raw and starting materials, that would be biopreserved and exclude potentially harmful enteropathogens. It would be further advantageous if the fermented feed could provide a probiotic effect to the animals receiving it and reduce or prevent illness of the animals due to infections and pathogenic challenge.

The inventors have now found that such a fermented liquid feed composition may be produced using one or more lactic acid bacteria possessing certain characteristics.

Accordingly, in a first aspect, the present invention provides a fermented liquid feed composition for a target animal, the feed composition being prepared by the fermentation of a feed substrate with a lactic acid producing bacteria, the lactic acid bacteria being characterised by:
a) being viable under the conditions prevailing in the gastrointestinal tract of the target animal;
b) being a bacteria capable of aggregating and/or coaggregating with one or more pathogens; and
c) being able to produce upon fermentation in the feed substrate lactic acid in an amount of at least a minimum inhibitory concentration of lactic acid.

It has been found that a fermented feed according to the present invention produced by the fermentation of a feed substrate with a lactic acid producing bacteria having the characteristics set out above provides significant advantages over known feeds. In particular, the feed is able to be stored for extended periods of time and be transported without spoiling, the fermented feed being resistant to the growth of mould and yeasts and resistant to invasion and colonisation by bacteria potentially harmful to the target animals and other livestock. Further, the fermented feed, once consumed, provides significant protection for the animals against infection by bacteria, in particular pathogenic bacteria likely to cause serious illness or death of the animal. The fermented feed of the present invention achieves this by enhancing the barrier function of the upper gastrointestinal tract of the target animal. This advantage is particularly significant in the feeding of newly born and newly weaned animals, and newly hatched birds. In the large scale rearing of animals, for example cattle, pigs and poultry, there are particular challenges when young animals are removed from their mothers (weaned) and reared in a different environment. The weaning process removes immunoglobulin support provided by the mother's milk and precipitates changes to the gut ecosystem. This, in turn, leaves the young animals open to infection with a wide range of potentially harmful microorganisms. The high mortality rate of young animals is at least in part due to animals succumbing to such infections of microorganisms. The fermented feed of the present invention reduces or eliminates this risk, by populating the gastrointestinal tract of the young animal with healthy, beneficial microorganisms, in turn providing protection of the young animals against infection by pathogenic microorganisms.

The fermented feed of the present invention is provided for a target animal, which may determine such factors as the composition of the feed and the particular bacteria employed in the fermentation of the feed substrate. The fermented feed of the present invention may be provided for a wide range of animals and livestock, including mammals and poultry. Examples of target mammals include all the mammals farmed or reared, including horses, sheep, goats, pigs, cattle and deer, as well as animals reared for fur, such as mink and the like. Examples of target poultry include all the birds reared and farmed on a commercial scale, including chickens, ducks, geese, quail and turkeys, as well as game birds, such as pheasants, partridges and the like. The fermented feed may also be provided to farmed and ornamental fish and crustaceans. Further, the fermented feed may be provided for animals kept as domestic pets, such as dogs, cats, rabbits and the like.

In one preferred embodiment, the fermented feed of the present invention is advantageously formulated for providing to poultry, including chickens, quail, turkeys, geese, ducks and the like.

In a second preferred embodiment, the fermented feed of the present invention is advantageously formulated for providing to mammals, in particular pigs and ruminants, such as cattle and sheep, particularly during the post-natal and pre-ruminant stage and veal calves, in which ruminant function my be delayed.

The fermented feed of the present invention may be provided to any age of target animal, from newly born animals or newly hatched birds to mature adult animals. The fermented feed has been found to be particularly advantageous when provided to newly born and newly weaned animals or newly hatched birss, where the fermented feed provides such advantages as increased weight gain of the young animals and a reduction in infection with potentially harmful or pathogenic microorganisms. This results in a reduction in the harmful or pathogenic microorganisms shed by the animals, in turn increasing the health of other animals being reared in the same environment and the ultimate consumer of the animal and/or its products.

The fermented feed is prepared from a feed substrate, which is inoculated with a culture containing the lactic acid producing bacteria and fermented. The thus inoculated and fermented substrate may itself form the finished feed. Alternatively, the substrate, once fermented may be added to other feed materials, in order to provide the other materials with the biopreservative effects.

The feed substrate may be any suitable substrate that may be consumed by the target animals in a fermented condition. The feed substrate may consist of a substrate from a single or source or, alternatively may comprise a combination of substrates.

Suitable substrates include organic materials, such as plants or plant material, for example, fibrous plant material, such as grass; cereals and grains, such as wheat, barley, maize, rice, sorghum and rye; whole crop cereals, maize silage and corn cob meal; root crops, such as potatoes, swedes, fodder beet, sugar beet and the like; pulses and seeds, such as beans, peas, soya bean and rapeseed (and their residues); brassiccas and the like. Further substrates include organic residues, such as materials produced as co-products or residues from dairy operations, such as whey, curd and skimmed milk, ice cream, yoghurt, off-specification butter and cheese; from the baking and confectionary industry, such as biscuit meals, cereal residues, misshapen and off-specification breads, cakes and biscuits; from the beverage industry, such as fruit pulps, grape pulp, coffee and chocolate residues; brewing and distilling residues; from cooking oil extraction, such as rapeseed meal, soya bean meal and olive pulp; from meat and fish slaughter and processing; or the production of bio-fuels.

To be suitable for fermentation, the feed substrate should contain water in an amount sufficient to support fermentation with the lactic acid producing bacteria. Preferably, the feed substrate has a water content of at least 20% by weight, more preferably at least 30% by weight, still more preferably at least 40% by weight. Preferred ratios of dry feed substrate to water are dependant on the target species to be fed and range from 1:0.25 to 1:4, more preferably from 1:0.4 to 1:2. One preferred ratio of dry feed substrate to water is about 1:1.2 for chickens and 1:2.5 for pigs. The precise water content of the feed substrate will be determined by such factors as the nature and composition of the feed substrate, the lactic acid producing bacteria being employed and the end use of the fermented feed and target animal. References herein to a ‘moist feed’ or ‘liquid feed’ are to a feed material containing at least the minimum water content to support fermentation of the feed by the lactic acid producing bacteria and the terms ‘moist feed’ and ‘liquid feed’ are to be understood and interpreted accordingly.

The feed substrate may contain sufficient water to support fermentation with the lactic acid bacteria. If not, water should be added to the feed substrate to achieve the water content required for fermentation.

To produce the fermented feed of the present invention, the feed substrate is inoculated with lactic acid producing bacteria and fermented. The lactic acid producing bacteria employed in the present invention are characterised by the following features:
a) The bacteria are viable under the conditions prevailing in the gastrointestinal tract of the target animal;
b) The bacteria are capable of aggregating and/or co-aggregating with one or more pathogens; and
c) The bacteria are capable of producing lactic acid upon fermentation with the feed substrate to at least a minimum inhibitory concentration in the fermented feed.

Bacteria having the three characteristics (a) to (c) give rise to the advantageous properties of the fermented feed of the present invention. As noted above, the feed substrate is inoculated with the lactic acid producing bacteria.

Accordingly, in a further aspect, the present invention provides an inoculant for the preparation of a fermented feed from a feed substrate, the inoculant comprising a viable culture of a lactic acid producing bacteria having the following characteristics:
a) being viable under the conditions prevailing in the gastrointestinal tract of the target animal;
b) being a bacteria capable of aggregating and/or co-aggregating with one or more pathogens; and
c) being able to produce upon fermentation in the feed substrate lactic acid in an amount of at least a minimum inhibitory concentration of lactic acid.

The inoculant may be in any suitable form and of any suitable composition so as to contain viable lactic acid producing bacteria for populating and fermenting the feed substrate. Preferred presentations for the inoculant are freeze dried or as a liquid culture.

The inoculant should contain the lactic acid producing bacteria in a viable form and in sufficient concentration to allow the feed substrate, once inoculated, to ferment and produce the required number of viable lactic acid producing bacteria and the required concentration of lactic acid in the fermented feed. A typical number of lactic acid producing bacteria in the inoculant is from 10⁵ to 10⁹ CFU/ml, more preferably about 10⁶ CFU/ml, if presented in liquid form or 10⁵ to 10⁹ CFU/g, more preferably about 10⁶ CFU/g if presented in freeze dried form.

For example, a suitable inoculant for a substrate is 0.1% of a liquid broth culture containing 10⁹ CFU/ml of the lactic acid producing bacteria or 0.1% of a freeze dried culture containing 10⁹ CFU/g of the lactic acid producing bacteria.

The inoculant organism may be presented in a suitable carrier to maintain shelf life and facilitate accurate dispersion when added to the substrate. Such methods are known in the art and readily understood by the person skilled in the art.

As a first characteristic, the lactic acid producing bacteria should be viable and survive in the gastrointestinal tract of the target animal. The conditions in the gastrointestinal tract of many animals are severe enough to prevent the colonisation and growth of many microorganisms. In particular, the upper gastrointestinal tract of many animals is sufficiently acidic to prevent many species of microorganisms from thriving and remaining viable. In order to provide the advantageous properties of the fermented feed of the present invention, the lactic acid producing bacteria used to ferment the feeds substrate should be viable under the acidic conditions prevailing in the upper gastrointestinal tract of the target animal and the alkaline conditions encountered in the duodenum, and should remain viable in both the small intestines and the large intestines. In addition, in the case of fermented feed intended for providing to poultry, the lactic acid producing bacteria should also remain viable under the conditions prevailing in the crop and proventiculus, as well as the gizzard.

The viability of the bacteria in the gastrointestinal tract may be determined by methods and techniques known in the art. In particular, the microbial count of the viable lactic acid bacteria in the faeces of target animals fed a diet containing viable lactic acid bacteria may be measured. Alternatively, the lactic acid bacteria count in the gastrointestinal tract of poultry fed a diet containing the bacteria may be determined using cloacal swabs. Such methods are known in the art and readily understood by the person skilled in the art.

As a further alternative or in addition thereto, the viability of the lactic acid producing bacteria in the gastrointestinal tract of the target animal may be determined in vitro, in particular by measuring the growth of the microorganisms under acidic conditions similar to or the same as those prevailing in the upper gastrointestinal tract of the target animal. Thus, in the case of a fermented feed intended for pigs, the viability of the lactic acid producing bacteria may be determined after exposure to pH 2 for 2 hours followed by buffering to pH 6.8 and exposure to bile salts for 4 hours in a suitable feed substrate, to represent conditions in the stomach and the small intestine of the target animal. The acidity of the large intestine is generally similar to that of the small intestine, meaning that viability in the large intestine is most likely for all microorganisms surviving under conditions of lower pH prevailing in the stomach. Similarly, viability in the gastrointestinal tract of poultry may be determined by sequentially exposing the lactic acid producing bacteria in a suitable feed substrate to a pH of from 4.4 to 4.5, as encountered in the crop and proventiculus, a pH of about 2.6, as encountered in the gizzard, and to a pH of 6.2 in the presence of bile salts as encountered in the small intestine of the target birds. Again, conditions in the caecum are unlikely to adversely affect organisms that survive the gizzard and small intestine.

In order to more accurately model the conditions of the gastrointestinal tract of the target animal, it is further preferred that the in vitro experiments described hereinbefore are conducted using the feed substrate of the eventual fermented feed as the growth medium for the microorganisms. In this way, any effects produced within the gastrointestinal tract of the target animal when fed with the fermented feed that may alter the conditions therein and/or the viability of the lactic acid producing bacteria may be determined.

A procedure for the in vitro determination of the viability of a lactic acid producing bacteria in the gastrointestinal tract of a target animal is described in detail in Example 1 hereafter.

In a particularly preferred embodiment, the fermented feed of the present invention comprises lactic acid producing bacteria that have been demonstrated to be viable in the gastrointestinal tract of the target animal using the aforementioned in vitro procedure employing the feed substrate as growth medium for the microorganisms.

As a second requirement, the lactic acid producing bacteria of the fermented feed of the present invention are aggregating bacteria, that is the bacteria form aggregates. In addition, or alternatively, the bacteria are capable of co-aggregating with other microorganisms, in particular microorganisms that are pathogenic to the target animal, that is form aggregates together with the other microorganisms. Preferably, the lactic acid producing bacteria are both aggregating and co-aggregating. The ability to aggregate and/or co-aggregate may be exhibited by the lactic acid producing bacteria under the conditions in the feed substrate during and after fermentation and/or under the conditions prevailing in the gastrointestinal tract of the target animal. Preferably, the bacteria are aggregating and/or co-aggregating both in the feed substrate and in the gastrointestinal tract of the target animal.

The ability of the microorganisms to aggregate in vitro gives a strong indication of their ability to adhere to the mucus layer in the gut and the epithelial cells of the intestinal wall of the target animal and, generally, to colonise the gastrointestinal tract. This in turn increases the resistance of the target animal to infection by exclusion of harmful or pathogenic microorganisms from attachment sites. Further, the lactic acid producing bacteria are preferably ones that are coaggregating, that is form coaggregations with other microorganisms, in particular harmful or pathogenic bacteria. In particular, it is preferred that the lactic acid producing bacteria are coaggregating with strains of Salmonella, E. Coli, and/or Clostridium. This in turn increases the passage and clearance of the harmful bacteria from the gut lumen.

The ability of a lactic acid producing bacteria to aggregate may be determined by in vitro methods and techniques known in the art, for example as described in Drago. L. et al., noted above. In particular, the bacteria may be cultured in a suitable liquid growth medium, such as Man-Rogosa-Sharpe (MRS) broth (available commercially). Bacterial aggregates may be identified as grains or particles that develop in the liquid culture medium, typically collecting at the bottom of the culture vessel under the action of gravity and leaving a clear supernatant liquid.

Similarly, the ability of the lactic acid producing bacteria to coaggregate with other bacteria may be determined by preparing a co-culture of the lactic acid producing bacteria with one or more target bacteria in like manner with the formation of aggregates being observed as grain-like particles that tend to settle in the culture, again leaving a clear supernatant liquid.

While the formation of aggregates and coaggregates in the bacterial cultures may be observed using the naked eye, as described above, further and more detailed information regarding the aggregating ability of the microorganisms may be obtained by using microscopy techniques, including scanning electron microscopy (SEM).

A procedure for the identification of lactic acid bacteria that are aggregating and coaggregating is set out in Example 2 below.

As a third characteristic, the lactic acid producing bacteria of the fermented feeds of the present invention are capable of producing at least a minimum inhibitory lactic acid concentration in the fermented feed. In respect of the fermented feeds of the present invention, the term ‘minimum inhibitory lactic acid concentration’ is a reference to a lactic acid producing bacteria that is capable of producing at least 150 mMol of lactic acid in 24 hours upon fermentation at 30° C. in a growth medium consisting of MRS broth containing 2% by weight glucose. It has been found that lactic acid producing bacteria that are capable of producing this minimum concentration of lactic acid in the aforementioned test are particularly advantageous in the preparation of fermented feeds and providing significant health benefits to the target animals provided with the feed.

The concentration of lactic acid in the culture medium may be determined using methods known in the art, for example the method of Niven, S. J., et al., ‘The simultaneous determination of short chain fatty acid monosaccharides and ethanol in fermented liquid pig diets’, Animal Feed Science and Technology, 117 (2004), (3-4), pages 339 to 345.

A procedure for identifying lactic acid producing bacteria capable of producing at least the minimum inhibitory lactic acid concentration is set out in Example 3 below.

More preferably, the lactic acid producing bacteria is capable of producing at least 200 mMols of lactic acid under the aforementioned procedure and test conditions, still more preferably at least 250 mMols of lactic acid. Lactic acid concentrations of at least 300 mMols, more preferably at least 350 mMols produced under the aforementioned test conditions may also advantageously be applied.

In general, a higher concentration of lactic acid in the fermented feed, and consequently a lower pH value for the fermented feed is to be preferred. Accordingly, preferably the pH value of the fermented feed is 4.5 or lower, more preferably 4.0 or lower, still more preferably 3.5. The lower limit of pH value and, hence, the upper limit for lactic acid concentration will be, at least in part, determined by the target animal and its ability and willingness to eat the fermented feed. As the pH is lowered further, the target animals may refuse to eat the feed.

The lactic acid producing bacteria employed to prepare the fermented feed of the present invention may be either homofermenting or heterofermenting. Heterofermenting bacteria produce lactic acid as a product of their metabolism, along with other organic acids, such as, for example, acetic acid, propionic acid and butyric acid. However, it has been found that the presence of significant quantities of these other acid metabolites may adversely affect the taste of the fermented feed and/or reduce the nutritional value of the feed to the target animal. In contrast, homofermenting lactic acid producing bacteria are ones that metabolise the feed substrate to produce lactic acid as the only acid metabolite. Accordingly, it is preferred that the lactic acid producing bacteria present in the fermented feed are homofermenting.

Further, the lactic acid producing bacteria used in the fermented feed of the present invention are preferably antagonistic towards pathogens common to the target animal. For example, in the case of fermented feed intended to be provided to poultry, it is preferred that the lactic acid producing bacteria have antagonistic activity against one or more strains of Salmonella, Clostridium and E. coli.

A procedure for determining the antagonistic activity of a lactic acid producing bacteria is set out in Example 4 below.

In addition, the lactic acid producing bacteria used in the fermented feed of the present invention are preferably capable of adhering to the epithelial cells of the gastrointestinal tract of the target animal. In vitro methods for determining the adhesion of bacteria in this manner are known in the art.

A procedure for determining the ability of the lactic acid producing bacteria to adhere to the epithelial cells of the target animal is set out in Example 5 below.

Suitable lactic acid producing bacteria for use in the fermented feed of the present invention are naturally occurring and may be isolated from suitable sources using techniques known in the art. Suitable sources of lactic acid producing bacteria for use in the present invention include the gastrointestinal tract of animals and birds, including but not limited to the gastrointestinal tract of the target animal or bird of the fermented feed concerned. Other sources of lactic acid producing bacteria include cereal grains, spontaneous fermentations in substrates, and the teats and other parts animals. Isolation of the lactic acid producing bacteria may be carried out using techniques known in the art.

Lactic acid producing bacteria may be identified again using techniques known in the art. For example, Lactobacilli may be identified using the gram stain and catalase tests, with Lactobacilli being gram positive and catalase negative rods.

In a further aspect, the present invention provides a method for preparing a fermented feed composition, the method comprising fermenting a feed substrate with a lactic acid producing bacteria, the lactic acid bacteria being characterised by:
a) being viable under the conditions prevailing in the gastrointestinal tract of the target animal;
b) being an aggregating bacteria and/or co-aggregating with one or more pathogenic bacteria; and
c) being able to produce upon fermentation in the feed substrate lactic acid in an amount of at least a minimum inhibitory concentration of lactic acid.

The fermented feeds of the present invention may be prepared in any suitable manner. Typically, the fermented feeds are prepared by inoculating the feed substrate with an inoculum containing the lactic acid producing bacteria in viable form and fermenting the feed substrate under suitable conditions. Techniques for fermenting a feed substrate after inoculation with a lactic acid producing bacteria are known in the art.

The feed composition being fermented contains water. If a dry feed substrate is being employed, water is added to the substrate. The feed composition being fermented preferably contains water in an amount of from 1 to 10 parts water by weight for each part of feed substrate (dry basis), more preferably from 1 to 5 parts water, by weight. One preferred embodiment comprises the feed substrate and water in a weight ratio of from 1:1 to 1:3, more preferably from 1:1 to 1:2, especially from 1:1 to 1:1.5.

Fermentation of the feed substrate may be conducted at any temperature suitable for the cultivation of the lactic acid producing bacteria. The optimum temperature for fermentation will depend upon the strain or strains of bacteria being employed. Typically, the feed substrate is fermented at a temperature of from 15 to 45° C., more preferably from 30 to 35° C.

The feed substrate is fermented for a sufficient period of time to allow the lactic acid producing bacteria to produce at least a minimum lactic acid concentration of 150 mMol/l lactic acid, more preferably at least 200 mMol/l, still more preferably at least 250 mMol/l. Typical fermentation times are from 8 to 72 hours, more preferably from 8 to 24 hours.

The production of lactic acid in the fermented feed may be monitored by measuring the pH of the feed composition, which will fall as lactic acid is produced during the fermentation process. The pH of the feed composition after fermentation with the lactic acid producing bacteria is preferably 4.5 or lower, more preferably 4.0 or lower.

As noted above, feeds having a low pH may be unpalatable to the target animals. Fermented feeds having higher concentrations of lactic acid and pH values below 3.5 may be advantageously combined with other materials to produce a final diet, as long as the minimum inhibitory concentration of lactic acid is maintained.

Nutrients and other components essential to the growth of the lactic acid producing bacteria may be added to the feed substrate, as required. Such nutrients and components will be known in the art.

The feed substrate is fermented to produce a concentration of lactic acid producing bacteria in the feed composition that is beneficial to the target animals. In particular, the lactic acid bacteria present in the feed composition after fermentation is completed should be viable in sufficient numbers to colonise the gastrointestinal tract of the target animal and form viable colonies therein. Preferably the concentration of lactic acid producing bacteria in the fermented feed is at least 10⁶ CFU/ml, more preferably from 10⁷ to 10¹⁰ CFU/ml, still more preferably from 10⁹ to 10¹⁰ CFU/ml.

The feed composition and method of the present invention may employ any suitable lactic acid producing bacteria, with the proviso that the bacteria is not harmful to the target animal. Preferred lactic acid producing bacteria include strains of Lactobacillus and Pediococcus, with strains of Lactobacillus being particularly preferred. Particularly preferred microorganisms of the strain Lactobacillus include strains of Lactobacillus plantarum and Lactobacillus salivarius.

Extensive work has been carried out to isolate a series of strains of Lactobacillus of particular advantage in the preparation of fermented feeds according to the present invention. The strains were isolated by the general method described hereinbefore and using the detailed method described below. Each of the isolated strains exhibited all three of the properties (a) to (c) described above, making them particularly suitable for use in the preparation of a feed composition according to the present invention. Each of the isolated strains has been deposited on 11 Feb. 2009, with the National Collections of Industrial and Marine Bacteria Ltd., Aberdeen, Scotland (hereafter ‘NCIMB’) and accorded the NCIMB accession numbers set out below. The deposits have been made pursuant to and in satisfaction of the requirements of the Budapest Treaty on the International Recognition of the Deposit of Microorganisms for the Purposes of Patent Protection.

Accordingly, in a further aspect, the present invention provides biologically pure cultures of the following microorganisms:

Lactobacillus plantarum, strain number C28, accession number NCIMB 41605;

Lactobacillus salivarius ss. Salivarius, strain number MS3, accession number NCIMB 41606;

Lactobacillus plantarum, strain number MS18, accession number NCIMB 41607;

Lactobacillus plantarum, strain number VD23, accession number NCIMB 41608;

Lactobacillus salivarius ss. Salivarius, strain number MS6, accession number NCIMB 41609; and

Lactobacillus salivarius ss. Salivarius, strain number MS16, accession number NCIMB 41610.

In a further aspect, the present invention provides a composition for the preparation of a fermented feed, the composition comprising one or more of the aforementioned microorganisms and a suitable carrier.

On a more general note, it has been found that the administration to target animals of lactic acid producing bacteria having the following characteristics:
a) being viable under the conditions prevailing in the gastrointestinal tract of the target animal;
b) being an aggregating bacteria and/or co-aggregating with one or more pathogens; and
c) being able to produce upon fermentation in the feed substrate lactic acid in an amount of at least a minimum inhibitory concentration of lactic acid;

is generally advantageous for the health and wellbeing of the target animal.

Accordingly, in a further aspect, the present invention provides a method for improving the general health of a target animal, the method comprising administering to the animal lactic acid producing bacteria having the following characteristics:
a) being viable under the conditions prevailing in the gastrointestinal tract of the target animal;
b) being an aggregating bacteria and/or co-aggregating with one or more pathogens; and
c) being able to produce upon fermentation in the feed substrate lactic acid in an amount of at least a minimum inhibitory concentration of lactic acid.

The microorganisms may be administered by way of the water provided to the animals, for example as a single dose or by continuous feeding with water containing the microorganisms. Alternatively, the microorganisms may be administered by way of the feed provided to the target animals, most preferably by way of a fermented feed as hereinbefore described.

The preferred lactic acid producing bacteria for general administering to target animals are as set out above.

The lactic acid producing bacteria are preferably administered to the target animal as viable microorganisms, preferably in a concentration of at least 10⁶ CFU/ml, more preferably at least 10⁷ CFU/ml, still more preferably in a concentration of at least 10⁹ CFU/ml. If administered to the target animal by way of its water, the minimum number of microorganisms is preferably at least 10⁶ CFU/ml. If administered by way of a fermented feed, the minimum number of lactic acid producing bacteria is preferably at least 10⁸ CFU/ml, more preferably up to 10¹⁰ CFU/ml.

It has been found that providing the target animals with lactic acid producing bacteria in this way increases the rate at which the animal increases in weight, and improves the overall health of the animal, in particular increasing the resistance of the animal to infection from potentially harmful microorganisms. This reduces the level at which the target animals shed harmful bacteria into their environment, in turn reducing the rate of infection of other animals in the vicinity of the target animals. These advantages have been found to be particularly marked when the lactic acid producing bacteria are provided to very young or immature animals.

Further, the present invention provides a biologically pure culture of a lactic acid producing bacteria having the following characteristics:
a) being viable under the conditions prevailing in the gastrointestinal tract of the target animal;
b) being an aggregating bacteria and/or co-aggregating with one or more pathogens; and
c) being able to produce upon fermentation in the feed substrate lactic acid in an amount of at least a minimum inhibitory concentration of lactic acid.

A composition for providing lactic acid bacteria to a target animal comprises a viable culture of the aforementioned lactic acid producing bacteria having characteristics (a) to (c) and a suitable carrier.

The present invention will be illustrated by way of the following specific examples and by reference to the accompanying figures, in which:

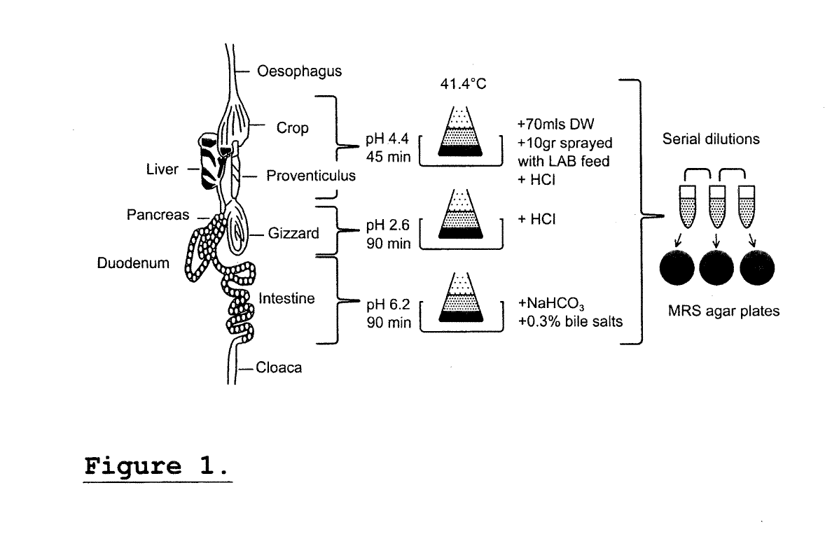

FIG. 1 is a diagrammatical representation of a preferred in vitro method of determining the viability of lactic acid producing bacteria in the gastrointestinal tract of the target animal;

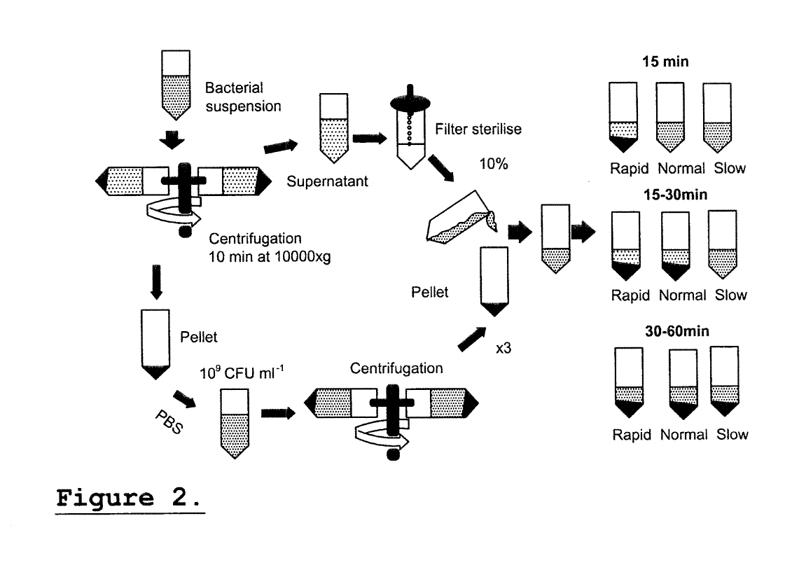

FIG. 2 is a diagrammatical representation of a preferred method for determining the aggregating and coaggregating ability of lactic acid producing bacteria; and

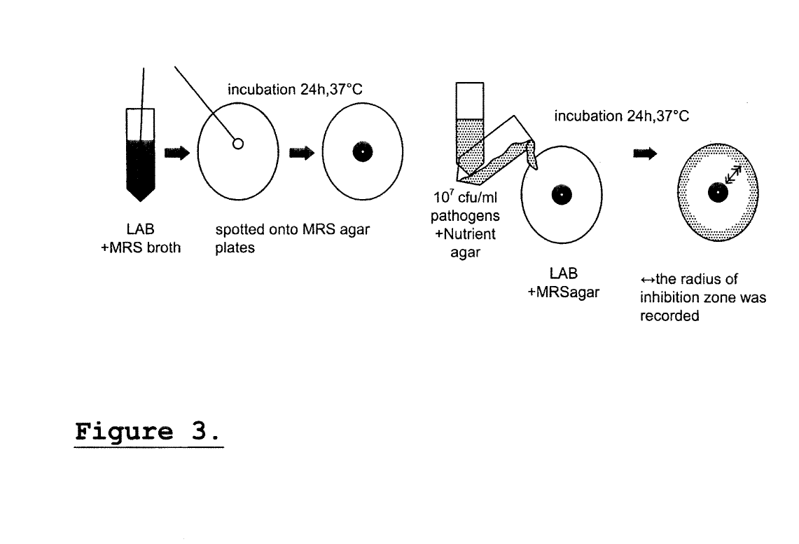

FIG. 3 is a diagrammatical representation of a preferred method for determining the antagonistic level of lactic acid producing bacteria.

The experiments described in the following examples were conducted using lactic acid producing bacteria sourced, isolated and identified as follows:

Three chickens (Hubbard breed; age 9 weeks and 2 days) were fed ad libidum on a diet of a commercially available organic growers ration, grass and clover. The chickens were humanely slaughtered and the entire gastrointestinal tract removed from each bird. Contents from the caecum, jejunum, ileum and crop were removed aseptically. In addition, epithelial cells were removed from the small intestine and the crop by scraping with a slide. All samples were diluted in 10 ml phosphate-buffered saline (PBS, ex. Oxoid, England) and plated in Man-Rogosa-Sharpe (MRS) and Rogosa agar (both ex. Oxoid, England). The streak method of isolation was used to obtain pure cultures from a mixed culture of bacteria. The thus isolated pure cultures were cultured for a second time in MRS agar plates and incubated in anaerobic jars in an atmosphere containing 5% vol carbon dioxide for 72 hours.

A total of 111 lactic acid producing bacteria were isolated on MRS agar (isolation medium for Lactocacillus and Pediococcus strains) and Rogosa agar (isolation medium for Lactobacillus strains).

Gram stains and catalase tests were used to confirm that the isolates were lactic acid producing bacteria. Isolates that were Gram positive and catalase negative were further identified by differential carbohydrate metabolism using API CHL kits (ex. BioMereux, UK).

The lactic acid producing bacteria thus isolated and identified as such were subjected to analysis using the procedures of Examples 1 to 3 to identify those meeting the requirements of:
a) being viable under the conditions prevailing in the gastrointestinal tract of the target animal;
b) being an aggregating bacteria and/or co-aggregating with one or more pathogens; and
c) being able to produce upon fermentation in the feed substrate lactic acid in an amount of at least a minimum inhibitory concentration of lactic acid.

In addition, the antagonistic activity of the isolated lactic acid producing bacteria against key pathogenic bacteria was determined following the procedure set out in Example 4. Further, the ability of the bacteria to adhere to epithelial cells was determined using the procedure set out in Example 5.

### EXAMPLES

### Example 1

### Determination of Viability of Lactic Acid Producing Bacteria in the Gastrointestinal Tract of the Target Animal

The viability of strains of lactic acid producing bacteria in the gastrointestinal tract of chickens was determined using the following procedure, which is summarised in FIG. 1:

Each strain of lactic acid producing bacteria was sprayed onto a commercially available pelleted poultry grower feed (ex. Mole Valley Farmers, Devon, UK). Prior to spraying with the bacteria, the feed was sterilised by irradiation (25 kGy, Co⁶⁰). The composition of the pelleted feed was as follows:

TABLE 1

| | Composition (% weight on a dry |
|---|---|
| Component | basis) |
| Barley | 4.97 |
| Wheat | 55.00 |
| Sunflower | 9.00 |
| Wheatfeed | 20.00 |
| Argentinean Soya | 0.50 |
| NGM Hi-pro Soya | 7.10 |
| Limestone Flour | 1.50 |
| Di-calcium Phosphate | 0.63 |
| Salt | 0.25 |
| Poultry GP Mins | 1.00 |
| Methionine | 0.05 |

A sample of the inoculated feed was added to a flask, diluted with the addition of distilled water and heated in a water bath to 41.4° C., to represent the temperature within the gastrointestinal tract of a chicken. The pH of the sample of the feed composition was adjusted successively by the addition of HCl (aq; 1M) to adjust the pH in the flask to correspond to the pH found at the successive stages in the digestive tract of poultry: pH 4.4 to 4.5 to correspond to the crop and proventiculus; pH 2.6 to correspond to the gizzard; and pH 6.2 corresponding to the small intestine. The sample was incubated at each pH for a period of time corresponding to the time digesta take to pass through the corresponding portion of the gastrointestinal tract: 45 minutes for the crop and proventiculus; 90 minutes for the gizzard; and 90 minutes for the small intestine.

HCl (aq; 1M) was added periodically to each sample throughout the incubation period, in order to maintain the pH at the appropriate level and counteract the normal buffering action of the feed components.

Samples (1 ml) of the solution in the flask was removed immediately before the pH was adjusted at each stage in the incubation, diluted with sterile peptone water (9 ml) and 10 fold serial dilutions were prepared. 100 μl of each dilution were spread over MRS agar using aseptic techniques and the plates incubated at 37° C. for 24 hours, after which the plates were counted. The viability of the microorganisms was calculated as the percent of organisms surviving passage through the simulated GI tract.

### Example 2

### Determination of Lactic Acid Bacteria that are Aggregating and Coaggregating

The ability of the lactic acid bacteria strains to aggregate and form coaggregates with other bacteria was determined using the following procedure, as illustrated in FIG. 2:

Lactic acid producing bacteria were grown overnight in MRS broth (ex Oxoid) at 37° C. in an atmosphere of 5% vol carbon dioxide. Thereafter, the cultures were centrifuged for 10 minutes at 10000 times gravity and washed three times with sterile distilled water. The thus washed material was resuspended in the same volume of phosphate-buffered saline (PBS) at a concentration of 10⁹ CFU/ml at a pH of 6.0 and incubated at room temperature.

Autoaggregation was determined to occur when clearly visible, sand-like particles were formed by the aggregated cells and gravitated to the bottom of the tubes within 2 hours.

In addition, the ability of the lactic acid producing bacteria to co-aggregate with other bacteria was determined by the following procedure:

The lactic acid producing bacteria were grown at 37° C. in MRS broth for 24 hours in an atmosphere containing 5% vol carbon dioxide. Salmonella spp. and E. coli were grown at 37° C. in nutrient for 24 hours in an atmosphere containing 5% vol carbon dioxide. Further. Clostridium perfringens were grown in clostridial broth for 24 hours under anaerobic conditions at 37° C. The following day, each culture was centrifuged for 10 minutes at 10000 times gravity and washed three times with sterile distilled water. The pathogenic cultures were resuspended in phosphate-buffered saline (PBS) to the same initial volume at a concentration of 10⁹ CFU/ml (ph 6.0) and incubated at room temperature in the presence of 10% vol freshly prepared filter-sterilised culture of the lactic acid producing bacteria supernatant liquid, at a total liquid volume of 1 ml. Coaggregation was taken as positive when clearly visible, sand-like particles formed by aggregated cells settled to the bottom of the vessel under gravity, leaving a clear supernatant liquid within a period of 2 hours.

Co-aggregation of the lactic acid producing bacteria with other potentially pathogenic microorganisms was tested in similar manner and confirmed using scanning electron microscopy.

### Example 3

### Determination of Lactic Acid Producing Bacteria Capable of Producing at Least the Minimum Inhibitory Lactic Acid Concentration

The ability of the lactic acid producing bacteria strains to produce lactic acid to at least the minimum inhibitory concentration was determined using the following procedure:

Lactic acid producing strains were grown in MRS broth for 24 hrs at 30° C. Ten ml aliquots of fresh MRS broth were inoculated with 0.1 ml of the 24 hr broth culture and incubated at 30° C. Subsamples of 1.0 ml were taken after 12, 24 and 48 hours for lactic acid analysis by high performance liquid chromatography according to the method of Niven et al. Standard MRS broth contains 2% glucose as a carbohydrate source giving a maximum lactic acid yield of 220 mMol/L.

### Example 4

### Determination of Antagonistic Activity of Lactic Acid Producing Bacteria

The antagonistic activity of the lactic acid producing bacteria strains with respect to pathogenic microorganisms was determined using the following procedure, as illustrated in FIG. 3:

The lactic acid producing bacteria were grown in MRS broth (ex. Oxoid, CM0359) at a temperature of 37° C. for 24 hours under anaerobic conditions. At the end of this period, samples of the bacteria were spotted onto MRS agar plates (ex. Oxoid) using a sterile cotton swab and incubated at 37° C. for a further 24 hours, again under anaerobic conditions, to allow colonies to develop. Nutrient agar containing approximately 10⁷ CFU/ml of each of five pathogenic bacteria Salmonella enteric Enteritidis (3 strains); Salmonella enteric Typhimurium (1 strain); and Escherichia coli (1 strain) was poured on the agar plate and the plate incubated for a further 24 hours at a temperature of 37° C. Nutrient agar containing approximately 10⁷ CFU/ml of Clostridium perfringens (1 strain) was poured on a second plate and incubated under anaerobic conditions at 39° C. for a further 48 hours.

At the end of the incubation periods, the plates were checked visually for inhibition zones around the Lactobacilli spots and the radius of the inhibition zone was recorded. A measure of the antagonistic activity of the subject lactic acid producing bacteria against the target pathogens may be obtained by measuring the radius of the inhibition zone around the lactic acid producing bacteria spot. A radius of from 1 to 2 cm indicated a high level of antagonistic activity.

### Example 5

### Determination of Adherence of Lactic Acid Producing Bacteria to Epithelial Cells

An experiment was conducted to determine the ability of strains of lactic acid producing bacteria to adhere to epithelial cells of organically farmed chickens using the following procedure.

The chickens were humanely slaughtered and ileal epithelial cells were removed by scraping the epithelium with a microscope slide. The cells thus removed were suspended in PBS and examined to ensure that they were free from any adherent bacteria. A haemocytometer was used to determine the number of cells.

Selected Lactobacilli were cultured overnight in MRS broth to give bacterial count of 10⁹ CFU/ml, and resuspended in PBS to give a cell density of 10⁸ CFU/ml. 100 μl of the Lactobacillus suspension was added to 400 μl of the epithelial cell suspension and the mixture incubated for 30 minutes at 37° C. with shaking. Adhesion of the Lactobacillus cells to the epithelial cells was observed using a phase contrast microscope by counting the number of bacterial cells adhered to epithelial cells selected at random from the resulting suspension.

### Example 6

An experiment was conducted to determine the benefits of treating chickens with strains of the lactic acid-producing microorganism Lactobacillus salivarius exhibiting the characteristics of a) being viable in the gastrointestinal tract of chickens (determined as outlined in Example 1); b) aggregating and co-aggregating with at least one or the following pathogens: Salmonella, E. coli, or Clostridia (determined as outlined in Example 2); and c) producing at least a minimum inhibitory concentration of lactic acid (determined as outlined in Example 3). In addition, the microorganisms were determined to be antagonistic to strains of Salmonella, Clostridium and E. coli using the method set out in Example 4. Using the procedure set out in Example 5, the lactic acid-producing bacteria were also determined to be highly adherent to chicken epithelial cells.

The strain of Lactobacillus salivarius employed was strain C28 referred to above.

Throughout the experiment, the birds were fed on a diet of clean water and a commercially available feed (Saracen Chick Crumbs, ex. J&W Attlee, Dorking, England). The feed had a moisture content of 14.0 wt %, with the composition, on a dry basis, as set out in Table II.

TABLE II
Composition of Feed

| Component | Composition (% wt on a dry basis) |
|---|---|
| Wheat | 54.5 |
| Hipro Soya | 16.7 |
| Barley | 10.0 |
| Minerals | 2.7 |
| Peas | 2.5 |
| Fishmeal | 1.5 |
| Vegetable fat | 1.2 |
| Vitamins | 0.75 |
| Methionine | 0.13 |
| Lysine | 0.03 |

102 specific pathogen-free chickens were randomly allotted to six groups of 17, with each group being treated as follows:

Group I: birds fed clean water and feed according to Table I.

Group II: birds treated by oral gavage at age 1 day with an aqueous medium containing Lactobacillus salivarius in a concentration of 10⁷ cfu/ml. Thereafter, the birds were fed as for Group I.

Group III: birds fed water containing 10⁷ cfu/ml Lactobacillus salivarius and feed according to Table I from age 1 day.

Group IV: birds fed water containing 10⁷ cfu/ml Lactobacillus salivarius and feed according to Table I from age 7 days.

Group V: birds fed clean water and fermented wet mash from age 1 day. The fermented wet mash was prepared by inoculating a mixture of the feed of Table I and water (ratio of feed to water of 1:1.2) with Lactobacillus salivarius and fermenting for 24 hours at 30° C. to obtain a microorganism concentration of about 10⁹ cfu/ml. The feed had a mean pH before fermentation with Lactobacillus salivarius of 5.94 and a mean pH after fermentation of 4.42.

Group VI: birds fed clean water and fermented wet mash from age 7 days. The fermented wet mash was prepared by inoculating a mixture of the feed of Table I and water (ratio of feed to water of 1:1.2) with Lactobacillus salivarius and fermenting for 24 hours at 30° C. to obtain a microorganism concentration of about 10⁹ cfu/ml. The feed had a mean pH before fermentation with Lactobacillus salivarius of 5.94 and a mean pH after fermentation of 4.39.

Weight of Feed Consumed by Birds

The experiment was conducted for a period of six weeks. As a first indicator of the health of the birds, the weight of feed being consumed by the birds in each group was monitored. The average daily feed consumption of the birds in each group during weeks 3 to 6 of the experiment is set out in Table 2.

TABLE 2
Average Daily Feed Consumption of Birds (g/bird/day on a
dry feed basis)

| GROUP | Week 3 | Week 4 | Week 5 | Week 6 |
|---|---|---|---|---|
| I | 44 | 49 | 59 | 59 |
| II | 43 | 49 | 61 | 75 |
| III | 43 | 47 | 52 | 52 |
| IV | 47 | 57 | 66 | 74 |
| V | 75 | 124 | 136 | 151 |
| VI | 77 | 121 | 132 | 151 |

As shown in Table 2, birds in Groups V and VI provided with the fermented feeds consumed significantly higher quantities of feed than the birds in other groups, indicative of a higher general level of health for the birds in Groups V and VI. In general, the birds provided with the lactic acid producing bacteria, whether by way of water or fermented feed, consumed larger quantities of feed, compared with the control group I.

Weight Gained by Birds

In addition to the quantity of feed consumed, the average weight gain for the birds in each group was measured. The average daily weight gain of the birds in weeks 3 to 5 of the experiment is shown in Table 3.

TABLE 3
Average Daily Weight Gain (g/bird/day)

| GROUP | Week 3 | Week 4 | Week 5 |
|---|---|---|---|
| I | 12.9 | 16.1 | 17.5 |
| II | 13.1 | 16.7 | 16.9 |
| III | 13.8 | 16.8 | 18.1 |
| IV | 14.2 | 16.2 | 18.7 |
| V | 15.2 | 16.0 | 22.1 |
| VI | 14.2 | 16.5 | 18.4 |

As shown in Table 3, the average daily weight gain of the birds treated with Lactobacillus salivarius was generally higher than that of the untreated birds. In particular, the initial weight gain of all birds provided with the lactic acid producing bacteria was significantly higher than that of the control group I. Further, the birds in Groups V and VI fed on fermented feed exhibited significantly higher weight gain, in particular in week 5 of the experiment.

Salmonella Shedding and Infection of Birds

At the start of the experiment, a random sample of birds from each group was cloacally swabbed and their faecal contents analysed to confirm no infection by strains of Salmonella.

To determine the effectiveness of the treatment with Lactobacillus salivarius in preventing infection of the birds by other microorganisms, all birds in each group were challenged with a strain of Salmonella according to the following procedure:

At 15 days of age, all birds were dosed with Salmonella typhimurium by oral gavage using a dosing catheter to administer an aqueous medium containing 10⁶ cfu/ml of Salmonella microorganisms. The Salmonella organisms employed were a nalidixic acid resistant derivative (SL1344 nal^{r}; ex. Veterinary Laboratories Agency (VLA), Weybridge, UK). Immediately prior to dosing with Salmonella, the birds were dosed in like manner with a solution of sodium bicarbonate, so as to instantaneously neutralise the acidity in the crop of the bird. In this way, the barrier imposed in the upper gut of the birds by acids in the crop was removed, permitting access of the introduced Salmonella to the lower gut environment.

Cloacal swabs were taken from the birds immediately before challenge and at least twice a week after challenge for a period of 4 weeks. The content of Salmonella typhimurium in the swabbed material was determined. In addition, the Salmonella-content of the bird litter was determined for each group. For each group, the percentage of birds that were found not to be shedding salmonella was determined. The results are set out in Table IV.

TABLE IV
Salmonella shedding

| | PERCENTAGE OF BIRDS NOT |
|---|---|
| GROUP | SHEDDING SALMONELLA |
| I | 8 |
| II | 14 |
| III | 19.5 |
| IV | 23 |
| V | 64 |
| VI | 82 |

From Table IV it can be seen that, in general, administering lactic acid producing bacteria to the birds significantly reduced the tendency of the birds to shed Salmonella into their environment. The reduction in Salmonella of the birds provided with the fermented feed, that is Groups V and VI, is particularly marked.

Further, throughout the duration of the experiment, it was found that at all times, the Salmonella shedding exhibited by the birds was consistently and significantly lower in the groups fed with the fermented feeds, that is Groups V and VI.

To determine the level of infection of the birds in each group, two post-mortem enumerations of Salmonella typhimurium infestations of the birds were carried out, one at 4 weeks of age and one at 6 weeks of age. The method employed was as follows:

The birds were euthanized by cervical dislocation. Their liver, spleen, ileum and caeca were removed aseptically and placed in sterile PBS. Samples of each tissue thus collected were weighed, homogenised and subjected to serial 10 fold dilutions in PBS (0.1M; pH 7.2). The viable count of Salmonella typhimurium in each homogenate was determined by plating drops of the dilutions on BGA supplemented with nalidixic acid (15 μg/ml). 1.0 ml of residual homogenate was added to 10 ml Selenite enrichment broth, incubated for 24 hours at 3TC, and thereafter subcultered on BGA supplemented with nalidixic acid. Viable counts of bacteria were determined by plating on MRS agar and incubating in anaerobic jars for 48 hours at 3TC.

The results of the first and second post mortem tests are set out in Tables V and VI respectively.

TABLE V
Salmonella typhimurium counts for first post mortem test

| | CAECUM | ILEUM | SPLEEN | LIVER |
|---|---|---|---|---|
| | (log₁₀ CFU/g | (log₁₀ CFU/g | (log₁₀ CFU/g | (log₁₀ CFU/g |
| GROUP | tissue) | tissue) | tissue) | tissue) |
| I | 5.7 | 4.3 | 2.5 | 2.1 |
| II | 6.1 | 2.7 | 4.0 | 2.2 |
| III | 5.6 | 2.2 | 3.8 | 1.6 |
| IV | 5.2 | 2.0 | 4.4 | 3.3 |
| V | 2.2 | 0.8 | 0.6 | 0.4 |
| VI | 2.1 | 1.0 | 1.7 | 0.8 |

TABLE VI
Salmonella typhimurium counts for second post mortem test

| | CAECUM | ILEUM | SPLEEN | LIVER |
|---|---|---|---|---|
| | (log₁₀ CFU/g | (log₁₀ CFU/g | (log₁₀ CFU/g | (log₁₀ CFU/g |
| GROUP | tissue) | tissue) | tissue) | tissue) |
| I | 2.3 | 2.3 | 0.7 | Nd |
| II | 4.1 | 2.5 | 1.9 | 1.8 |
| III | 2.3 | 1.0 | Nd | 0.4 |
| IV | 2.7 | Nd | Nd | 0.2 |
| V | 1.1 | Nd | nd | 0.7 |
| VI | 1.0 | 0.3 | 0.5 | 0.7 |

Nd = not detected

From Tables V and VI it can be seen that, in general, providing the birds with lactic acid producing bacteria significantly reduced the count of Salmonella typhimuriam in the birds. The reduction in the count of Salmonella typhimuriam was most notable in the birds in Groups V and VI fed with the fermented feeds.

Lactobacillus salivarius Colonisation of Birds

For the birds euthanized in the tests described above, the count of Lactobacillus salivarius in the caecum and ileum of the birds was also determined, using the general procedure outlined above, in order to determine the efficiency of the lactic acid producing bacteria in colonising the gastrointestinal tracts of the birds. The results are set out in Table VII.

TABLE VII
Lactobacillus salivarius in GI tract of birds

| | CAECUM | | | ILEUM | | |
|---|---|---|---|---|---|---|
| | Post | | | Post | | |
| | Mortem | Post | | Mortem | Post | |
| | 1 | Mortem 2 | Mean | 1 | Mortem 2 | Mean |
| | (log₁₀ | (log₁₀ | (log₁₀ | (log₁₀ | (log₁₀ | (log₁₀ |
| | CFU/g | CFU/g | CFU/g | CFU/g | CFU/g | CFU/g |
| GROUP | tissue) | tissue) | tissue) | tissue) | tissue) | tissue) |
| I | 7.5 | 8.3 | 7.9 | 9.9 | 8.3 | 8.7 |
| II | 8.9 | 8.4 | 8.6 | 9.4 | 8.8 | 9.1 |
| III | 8.9 | 8.3 | 8.6 | 9.1 | 8.8 | 9.0 |
| IV | 8.8 | 8.4 | 8.6 | 9.1 | 8.1 | 8.6 |
| V | 9.6 | 9.5 | 9.5 | 9.7 | 9.2 | 9.5 |
| VI | 9.5 | 9.6 | 9.5 | 9.7 | 9.2 | 9.5 |

As shown in Table VII, the provision of the lactic acid producing bacteria to the birds in the water and the feed significantly increased the degree of colonisation of the gastrointestinal tract by the bacteria. The fermented feeds provided to the birds in Groups V and VI were particularly effective in increasing the concentration of lactic acid producing bacteria in the gastrointestinal tract of the birds.

As a general result, the fermented feeds were particularly effective in reducing the colonisation of the birds by Salmonella. This is of significant advantage in the production of foodstuffs for humans, were the prime concern of food producers is the elimination of food bore pathogens, such as Salmonella from food animals and their products. Surprisingly, it was found that improved resistance to colonisation with Salmonella was achieved by providing the birds with fermented feed only from the age of 7 days, and not from age 1 day.

What is claimed is:

1. A biologically pure culture of one or more lactic acid producing bacteria selected from the group consisting of:
Lactobacillus plantarum, strain number C28, accession number NCIMB 41605;
Lactobacillus salivarius ss. Salivarius, strain number MS3, accession number NCIMB 41606;
Lactobacillus plantarum, strain number MS18, accession number NCIMB 41607;
Lactobacillus plantarum, strain number VD23, accession number NCIMB 41608;
Lactobacillus salivarius ss. Salivarius, strain number MS6, accession number NCIMB 41609; and
Lactobacillus salivarius ss. Salivarius, strain number MS16, accession number NCIMB 41610.

2. The use of a lactic acid producing bacteria for the preparation of a fermented liquid feed composition wherein the lactic acid producing bacteria is selected from the group consisting of:
Lactobacillus plantarum, strain number C28, accession number NCIMB 41605;
Lactobacillus salivarius ss. Salivarius, strain number MS3, accession number NCIMB 41606;
Lactobacillus plantarum, strain number MS18, accession number NCIMB 41607;
Lactobacillus plantarum, strain number VD23, accession number NCIMB 41608;
Lactobacillus salivarius ss. Salivarius, strain number MS6, accession number NCIMB 41609; and
Lactobacillus salivarius ss. Salivarius, strain number MS16, accession number NCIMB 41610.

3. A composition for the preparation of a fermented liquid feed composition comprising one or more lactic acid producing bacteria selected from the group consisting of:
Lactobacillus plantarum, strain number C28, accession number NCIMB 41605;
Lactobacillus salivarius ss. Salivarius, strain number MS3, accession number NCIMB 41606;
Lactobacillus plantarum, strain number MS18, accession number NCIMB 41607;
Lactobacillus plantarum, strain number VD23, accession number NCIMB 41608;
Lactobacillus salivarius ss. Salivarius, strain number MS6, accession number NCIMB 41609; and
Lactobacillus salivarius ss. Salivarius, strain number MS16, accession number NCIMB 41610;
and a suitable carrier.

4. A fermented liquid feed composition comprising one or more lactic acid producing bacteria selected from the group consisting of:
Lactobacillus plantarum, strain number C28, accession number NCIMB 41605;
Lactobacillus salivarius ss. Salivarius, strain number MS3, accession number NCIMB 41606;
Lactobacillus plantarum, strain number MS18, accession number NCIMB 41607;
Lactobacillus plantarum, strain number VD23, accession number NCIMB 41608;
Lactobacillus salivarius ss. Salivarius, strain number MS6, accession number NCIMB 41609; and
Lactobacillus salivarius ss. Salivarius, strain number MS16, accession number NCIMB 41610.
